(19) United States
(12) Patent Application Publication
(10) Pub. No.: US 2010/0078033 A1
(43) Pub. Date: Apr. 1, 2010

(54) THERAPEUTIC CUSHIONING DEVICE

(76) Inventor: Jonathan Aikman, Toronto (CA)

Correspondence Address: David B. Tingey, 1800 Eagle Gate Tower, 60 East South Temple, Salt Lake City, UT, 84145, US

(21) Appl. No.: 12/546,620

(22) Filed: Aug. 24, 2009

Related U.S. Application Data
(63) Continuation-in-part of application No. 12/091,170, filed on Apr. 22, 2008, now Pat. No. 7,578,013.
(60) Provisional application No. 60/730,456, filed on Oct. 26, 2005.

(51) Int. Cl. A61G 15/00; A61F 5/00

(52) U.S. Cl. 128/845; 602/18

(57) ABSTRACT

A therapeutic cushioning device that encourages its user to sleep on the user's right or left side, instead of on the user's back or chest. Generally, the cushioning device includes a head support, a torso support, a leg support, and a hinge member that movably connects the leg support to the torso support. In some instances, the head support includes one or more air tube management system notches to accommodate a facemask. In some instances, the torso support extends from the head support to form an L-shaped cushion. Moreover, the torso support optionally has a perimeter having a concave portion and convex portion, where the concave portion is placed closer to the head support than the convex portion. By encouraging the user to lie in a side position, the cushioning device can help the user avoid challenges associated with sleeping on the user's back or chest.

### RELATED APPLICATIONS

This application claims priority to and is a continuation-in-part application of U.S. patent application Ser. No. 12/091,170, which was filed on Apr. 22, 2008 and entitled THERAPEUTIC POSITIONING DEVICE, which is a 371 of international PCT/CA06/01748 that was filed on Oct. 25, 2006, which claims the benefit of U.S. Provisional Application No. 60/730,456, which was filed on Oct. 26, 2005; the entire disclosures of which are incorporated herein by reference.

### BACKGROUND OF THE INVENTION

1. Field of the Invention

The present invention relates to cushioning devices, such as pillows. In particular, the present invention relates to therapeutic cushioning devices that encourage a user to sleep on the user's right or left side, instead of on the user's back or chest. Generally, the cushioning device comprises a head support, a torso support, a leg support, and a hinge member that movably connects the leg support to the torso support.

2. Background and Related Art

Pillows are often used to support a user's head while the user is resting or sleeping. Indeed, pillows have long been recognized as an important element to obtaining restful sleep.

Many pillows are intended to help their users become comfortable in a variety of resting positions. For instance, some pillows are intended to help their users feel comfortable on the users' back (in a supine position), on the users' chest (in a prone position), and on the users' right or left side (in a side position).

It has been theorized that sleeping in a supine or prone position can have detrimental effects on some users. For example, sleeping in a supine or prone position may increase a user's likelihood of snoring, tend to restrict the user's airway, increase the user's likelihood of choking on vomit, and otherwise make sleeping less effective than if the user were in a side position.

In an effort to discourage people from sleeping in supine or prone positions, some have placed uncomfortable objects, such as tennis balls, near a person's back. For instance, some have sewn, strapped, or otherwise connected uncomfortable objects to pajama tops so that the user feels uncomfortable in a supine position. In other instances, some have made pillows of various shapes that allow their user to feel comfortable in a particular position.

While many conventional pillows help increase their user's comfort while resting or sleeping, such pillows are not without their shortcomings. By way of example, certain pillows do not encourage users to sleep in a side position. Accordingly, such pillows may do little to nothing to help prevent their users from experiencing the negative aspects associated with sleeping in a supine or prone position.

In another example, some pillows do little to make the use of a continuous positive airway pressure (“CPAP”) machine more convenient. For instance, when a user wearing a CPAP facemask for sleep apnea lies with a side of the user's face against a pillow, some pillows may apply pressure to the facemask and cause the user to find that position uncomfortable. Furthermore, sleeping with a facemask pressing against a pillow may result in abrasions or sores. As a result, such pillows may encourage their users to sleep in supine positions. However, because the user's airway can be restricted when the user is in a supine position, the air pressure from a CPAP machine may be need to be increased, which may make use of the CPAP machine even more uncomfortable. Accordingly, such pillows that encourage users to sleep in supine positions may actually discourage their users from using a CPAP machine altogether. This is an important development as compliance rates with CPAP and related therapies, such as BIPAP, are notoriously low, and challenging for physicians to treat.

In still another example, some pillows are resilient to the extent that a user who moves such a pillow from its natural position is required to hold the pillow in the new position to prevent it from returning to its natural position. Thus, such pillows may either slip away from the user as the user rests, or require the user to hold them in such a manner that reduces the user's ability to relax.

Thus, while techniques currently exist that are used to support a user's body, challenges still exist, including those previously mentioned. Accordingly, it would be an improvement in the art to augment or even replace current techniques with other techniques.

### SUMMARY OF THE INVENTION

The present invention relates to cushioning devices, such as pillows. In particular, the present invention relates to therapeutic cushioning devices that encourage a user to sleep on the user's right or left side, instead of on the user's back or chest.

Implementation of the present invention takes place in association with a therapeutic cushioning device that acts as a body support pillow. Generally, the cushioning device comprises a head support, a torso support, and a leg support, with hinge member movably connecting the leg support to the torso support.

With respect to the head support, this support can comprise any pillow that is capable of supporting a user's head. Some non-limiting examples of suitable head supports include pillows comprising natural fiber, synthetic fiber, foam, down, air, fluid, and/or combinations thereof. In some preferred implementations, the head support comprises a main core made from a first type of foam. In such instances, a layer of a second type of foam, which is softer than the foam of the main core, is optionally disposed over all or a portion of a front face and/or a back face of the head support.

The head support can have any other suitable characteristic that allows it to cushion the user's head. In one example, when the head support is at rest, meaning when the support is in its natural position or at its lowest potential energy state, the head support defines at least one air tubing management system (“ATMS”) notch at a perimeter of the support. This ATMS notch is generally sized so that the user can wear a facemask, such as an oxygen mask or a CPAP mask, and rest a side of the user's face on the head support without the facemask pressing against the support.

In another example, when the head support is at rest, it defines a head channel in the front and/or the back face. In this example, the head channel can be sized and positioned to support or cradle the user's head. While this channel may have any suitable characteristic, in some implementations, the channel extends from a first perimeter of the head support, across the length of the support, and to a second perimeter, which is disposed opposite to the first perimeter.

In still another example, when the head support is at rest, it defines a shoulder notch in at least one of the support's perimeters. In this example, when the user's head is placed on the head support, the user's shoulder need not lie on top of the head support. While the shoulder notch may perform many functions, in some instances, it helps straighten the user's neck when the user is resting on the head support.

Regarding the torso support, this support can have any suitable characteristic that allows it to support a portion of the user's body that is between the user's neck and the user's bottom, when the user's head is positioned on the head support and the user's torso is positioned adjacent to the torso support. In one example, a perimeter of the torso support comprises both a convex portion and a concave portion, wherein the concave portion is disposed closer to the head support than the convex portion. While these concave and convex portions can serve any suitable purpose, in some methods of use, the concave surface is configured to support the user's upper back while the convex portion is configured to support the user's lower back.

With respect to the hinge member, this member can comprise any suitable characteristic that allows it to movably connect the leg support to the torso support. Some non-limiting examples of suitable hinge members include a pillow case, a stuffed compartment, a strap, and any other suitable device that movably connects the leg support to the torso support. In preferred implementations, however, the hinge member is more flexible than the torso support. Thus, in such implementations, the hinge member allows the placement of the leg support to be easily adjusted and to remain in the adjusted position, without requiring the user to hold the leg support in the adjusted position.

Regarding the leg support, this support can comprise virtually any suitable characteristic that allows it to cushion one or more portions of the user's leg or legs, including, but not limited to, the user's thighs, knees, ankles, and/or feet. In some implementations, the leg support comprises an elongated block having a front and a back face. In such implementations, when the leg support is at rest, the leg support optionally comprises one or more indentations that are configured to receive one or more portions of the user's legs. For instance, the leg support can comprise an indentation in both its front face and its back face, wherein the indentations extend along the length of the leg support. In such instances, the indentations can help position the user's legs and relieve pressure at pressure points.

In addition to the aforementioned characteristics, the described cushioning device can comprise any other suitable component or characteristic. Indeed, in one example, the cushioning device comprises a pillow case. In this example, the pillow case can comprise any suitable characteristic. In some instances, the pillow case includes one or more openings that allow components of the cushioning device to be removed and be interchanged. In other instances, the pillow case comprises one or more ATMS connectors, such as straps with hook-and-loop fasteners, for retaining tubing for a facemask in certain positions with respect to the cushioning device.

The described cushioning device can also be modified in any suitable manner. In one example, one or more of the supports is configured to be selectively inflated and deflated. Accordingly, the cushioning device can be easily transported and the firmness or one or more of the supports can be adjusted as desired.

While the methods and processes of the present invention have proven to be particularly useful in the area of sleep apnea treatment, those skilled in the art can appreciate that the methods and processes can be used in a variety of different applications and in a variety of different areas to improve user comfort while resting. For example, the described cushioning device may be used to reduce snoring; to alleviation of back pain, neck pain, hip pain, leg pain, osteoarthritis pain, etc.; to reduce restless leg syndrome; to improve user blood flow; and for a variety of other purposes.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1A illustrates a front plan view of a representative embodiment of a therapeutic cushioning device;

FIGS. 1B-1G illustrate different views of some representative embodiments of the cushioning device;

FIG. 2A illustrates a cross-sectional view of a right and/or a left side of a representative embodiment of a head support;

FIGS. 2B-2E illustrate front plan views of some representative embodiments of the head support;

FIGS. 2F-2I illustrate different views of some representative embodiments of the head support;

FIG. 3A illustrates an exploded perspective view of a representative embodiment of the head support, a torso support, and a leg support from a representative embodiment of the therapeutic cushioning device;

FIG. 3B illustrates a plan view of a front face of a representative embodiment of the torso support;

FIGS. 3C and 3D respectively illustrate end views of a right side and a left side of the torso support from FIG. 3B;

FIG. 4 illustrates a front plan view of a representative embodiment the cushioning device;

FIG. 5A illustrates a face view of a representative embodiment of the leg support;

FIG. 5B illustrates a plan view of a left and a right side of the leg support from FIG. 5A;

FIGS. 6A-6D illustrate front plan views of some representative embodiments of a pillow case that is suitable for use with the therapeutic cushioning device;

FIGS. 7A-7B illustrate front plan views of some representative embodiments of the cushioning device;

FIGS. 8A-8C illustrate front plan views of some representative embodiments of the cushioning device;

FIG. 9 illustrates a front plan view of a representative embodiment of the therapeutic cushioning device, wherein the head support, the torso support, the leg support are selectively inflatable and deflatable; and

FIG. 10 illustrates a perspective view of a user resting on a representative embodiment of the therapeutic cushioning device.

In the drawings and in the specification, the use of the descriptive terms top, bottom, end, right, left, front, and back are used to identify portions of the elements illustrated in the drawings—while the elements are in the orientations shown in the drawings. It should be recognized that if the elements in the drawings were to be moved from their current orientations, these descriptive terms may refer to different portions of the elements shown. Accordingly, the use of these terms with respect to the drawings and in the specification is not meant to be limiting in any manner.

### DETAILED DESCRIPTION OF THE INVENTION

The present invention relates to cushioning devices, such as pillows. In particular, the present invention relates to therapeutic cushioning devices that encourage a user to sleep in a side position, instead of a supine or prone position.

As used in the description and in the claims, the term at rest and variations thereof may refer to a natural position or a position in which an object is at its lowest potential energy state.

The described therapeutic cushioning device comprises a body support pillow that encourages resting and/or sleeping in a side position. Accordingly, the described cushioning device may help to increase user comfort while resting or sleeping as well as to improve the effectiveness of the user's rest/sleep in a variety of manners. For example, by encouraging lying in a side position, the cushioning device can help reduce sleep apnea events that are associated with supine and prone sleeping positions. In some additional examples, by encouraging sleeping in a side position, the cushioning device can help to reduce snoring; alleviate pain, such as lumbar pain, knee pain, arthritic pain, post-surgical pain, chronic pain, muscle pain, joint pain, etc.; position the user in a position that increases the comfort of facemask use; alleviate restless leg syndrome; treat or prevent jaw disorders; treat or prevent bed sores; position handicapped users; improve blood flow (e.g., for diabetics); reduce the risks associated with users who have difficulty breathing; reduce post-anesthesia vomiting and aspiration; reduce risks for those who may generally be at risk of vomiting, aspirating or having their airway close in their sleep or rest; increase comfort for users suffering from fibromyalgia; and otherwise help users to be comfortable and obtain rest.

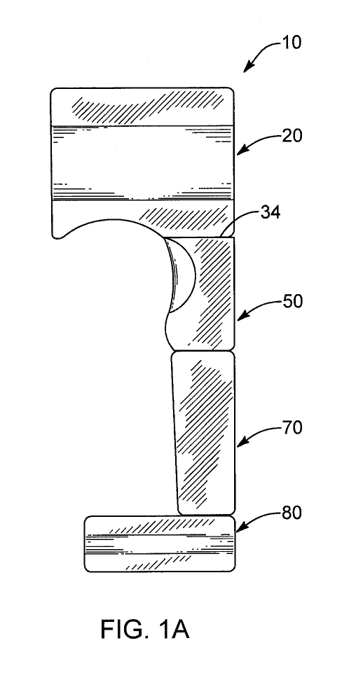

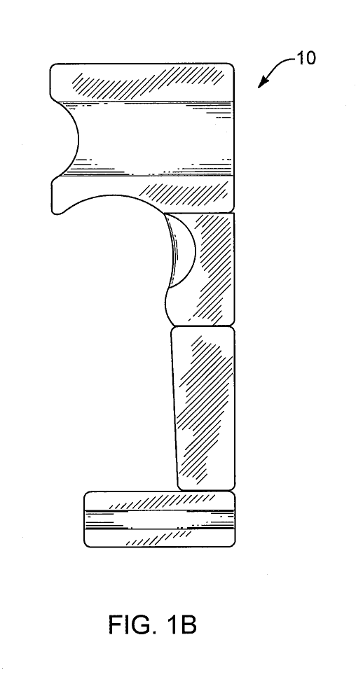

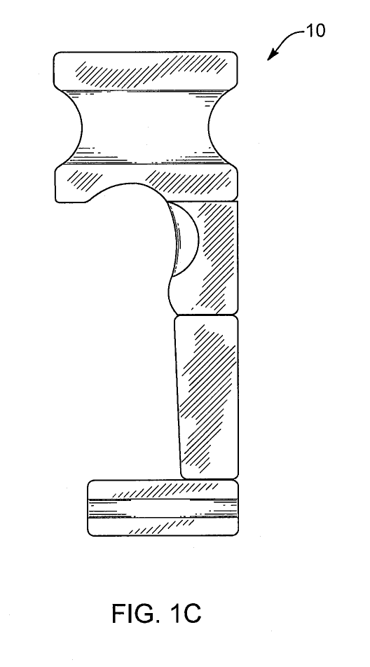

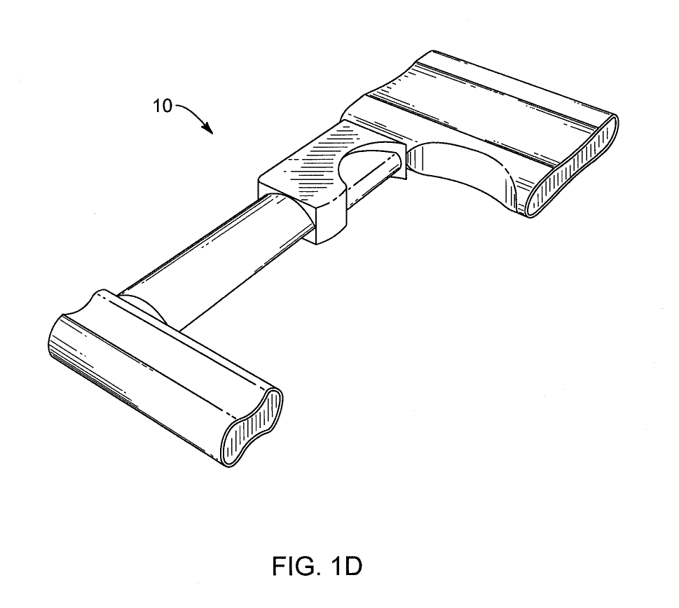

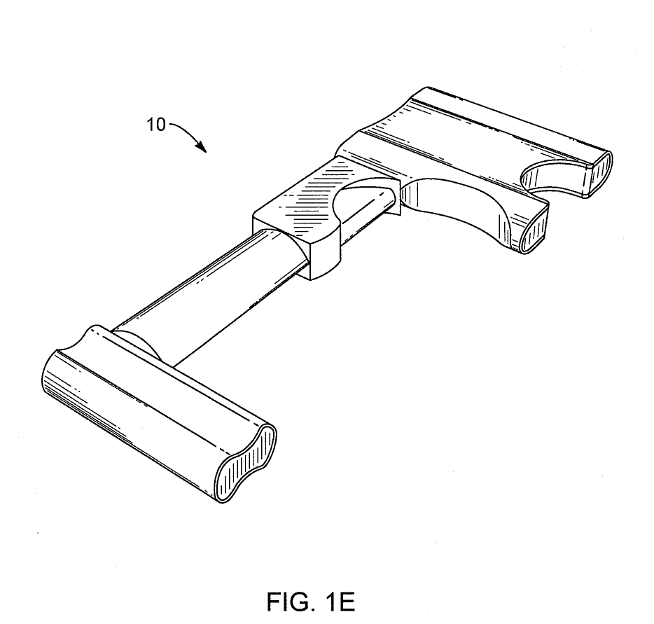

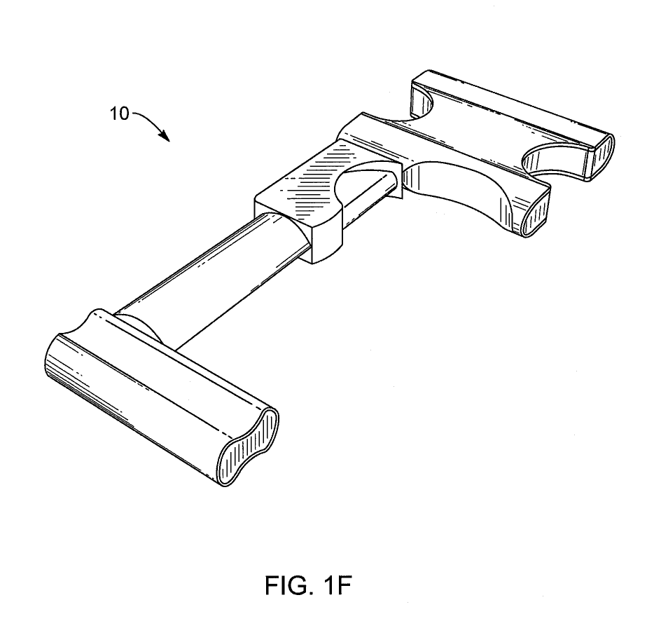

The described cushioning device may comprise any suitable component that allows it to encourage its user to sleep in a side position. By way of illustration, FIGS. 1A through 1G show several non-limiting embodiments of the therapeutic cushioning device 10. Specifically, FIG. 1A shows an embodiment in which the cushioning device 10 comprises a head support 20, a torso support 50, a hinge member 70, and a leg support 80. To provide a better understanding of the cushioning device, the aforementioned components are described below in more detail.

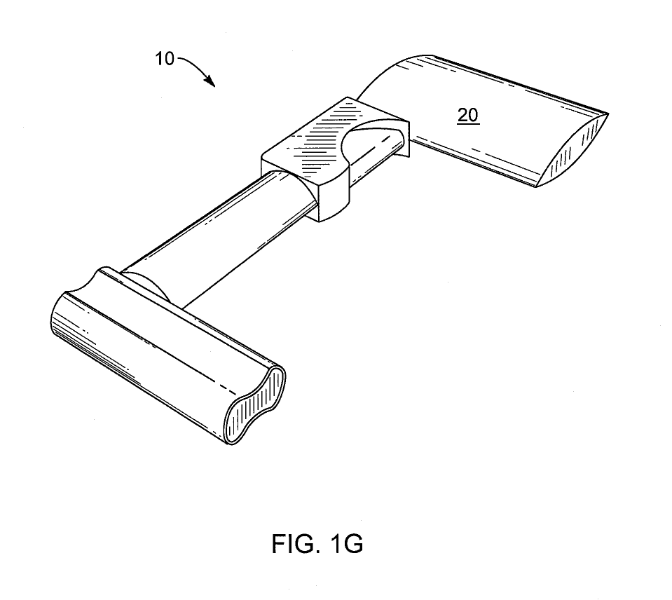

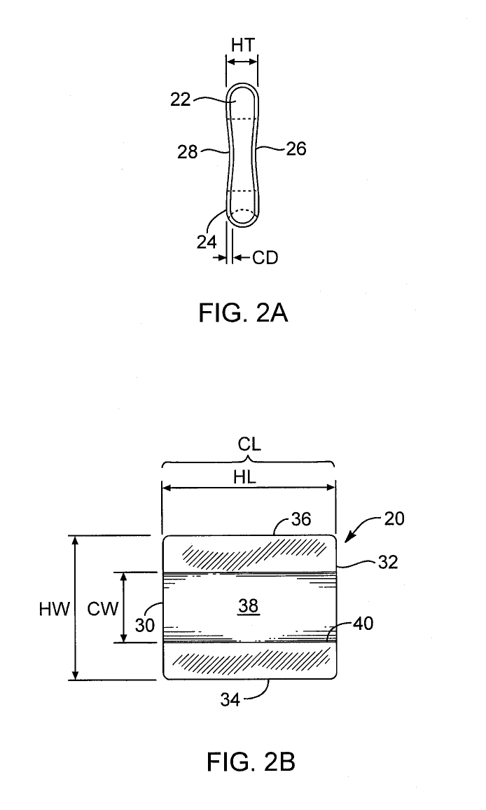

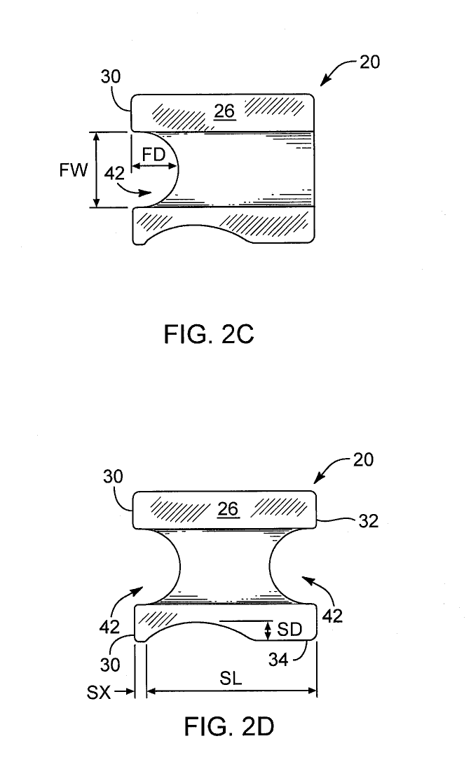

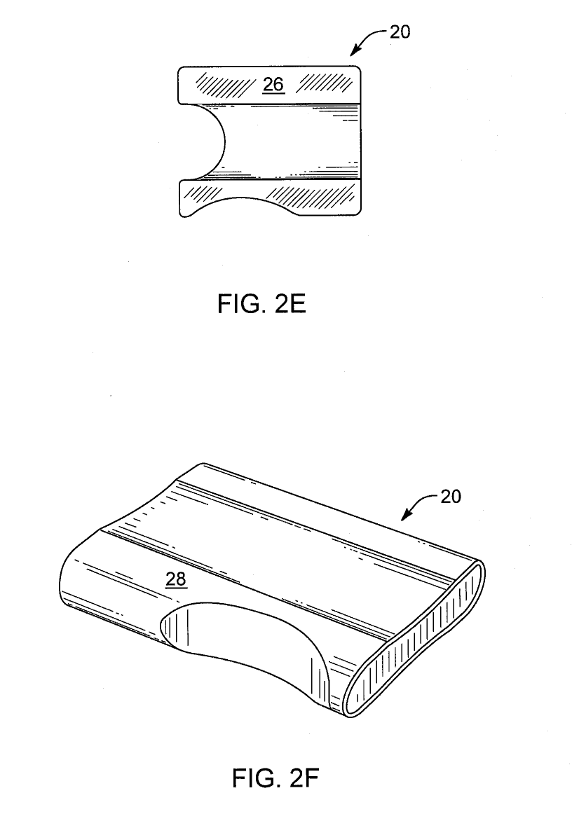

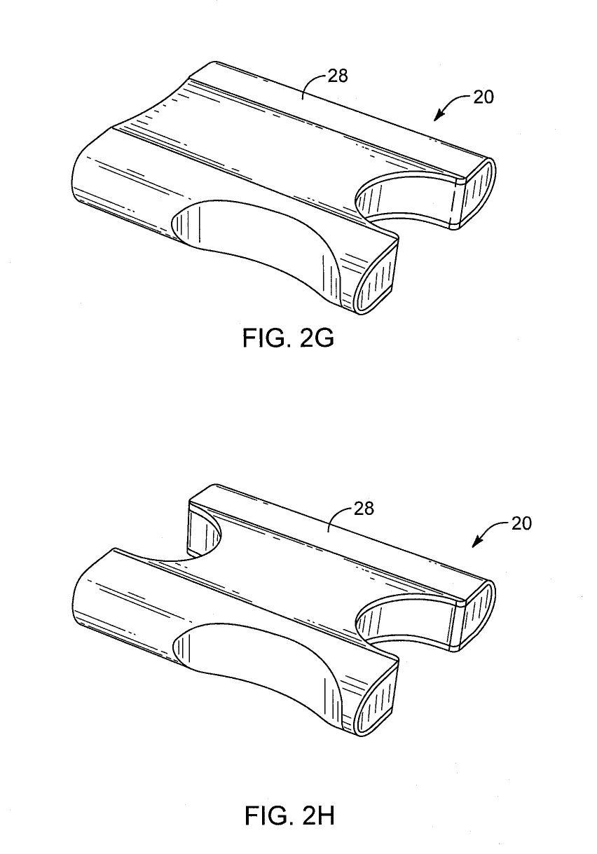

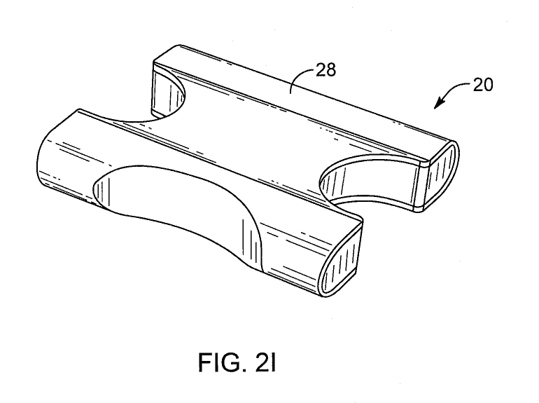

With respect to the head support, this support can comprise any pillow that is capable of supporting and cushioning the user's head. Some examples of suitable head supports comprise pillows that are filled with natural fiber, synthetic fiber, foam, down, air, fluid, other suitable pillow fillers, and combinations thereof. In some presently preferred embodiments, the head support comprises one or more types of foam. By way of illustration, FIG. 2A, which depicts a side view of the head support 20, shows an embodiment in which the support 20 comprises a main core 22 comprising a first type of foam, such as polyethylene foam. A layer of a second type of foam, such as visco-elastic foam (“memory foam”), which is softer than the first foam type, is optionally disposed over a portion, or all, of the exterior surface of the main core. For example, FIG. 2A shows an embodiment in which a layer 24 of the second type of foam is disposed over the main core 22.

FIG. 2A shows the head support 20 comprises a front face 26 and a back face 28. The front face 26 of the head support 20 (depicted in FIGS. 2B through 2I) can have any suitable shape, including, but not limited to, a square, rectangular, circular, elliptical, polygonal, or irregular shape. That said, FIG. 2B show that, according to at least one embodiment, the head support 20 is generally square or rectangular. Moreover, FIG. 2B shows that from its face view, the head support 20 comprises a first perimeter, or edge, 30 that is disposed substantially opposite to a second perimeter 32, and a third perimeter 34 that is disposed substantially opposite to a forth perimeter 36.

Where the head support is generally square or rectangular, the support can have any suitable dimensions that allow it to function as intended. In some embodiments, the length of the head support 20 (shown as HL in FIG. 2B) is between about 12 and about 36 inches. In other embodiments, the length HL of the head support is between about 20 and about 28 inches. In still other embodiments, the length HL of the head support is between about 22 and about 26 inches. Similarly, in some embodiments, the width of the head support 20 (shown as HW in FIG. 2B) is between about 10 and about 30 inches. In other embodiments, the width HW of the head support is between about 15 and about 25 inches. In still other more preferred embodiments, the width HW of the head support is between about 18 and about 22 inches.

Returning to FIG. 2A, that figure shows that the thickness HT between the lateral-most surface of the head support's front face 26 and the opposite lateral-most surface of the support's back face 28 can be any suitable distance. In some embodiments, the head support has a thickness HT between about 1 and about 8 inches. In other embodiments, the head support has a thickness HT between about 3 and about 7 inches. In still other embodiments, the head support has a thickness of between about 4 and about 6.5 inches thick. Of course the skilled artisan will recognize that the thickness of the head support, and any other support discussed herein, may vary depending on the support's firmness and/or the weight of the intended user.

In addition to the aforementioned characteristics, the head support can comprise any other suitable component or characteristic. In one example, when the head support is at rest, the support comprises an indentation or a head channel in the support's front and/or back face. In this example, the head channel can serve any suitable purpose, including cradling the user's face or head.

The head channel may be any suitable size. Indeed, in some embodiments, the head channel 38 has a length (shown as CL in FIG. 2B) that is between about 10 and about 15 inches. In other embodiments, the head channel has a length CL between about 14 and about 18 inches. In still other embodiments, FIG. 2B shows the head channel 38 has a length CL that is extends from the first perimeter 30 to the second perimeter 32 of the head support 20. Similarly, in some embodiments, the head channel has a width (shown as CW in FIG. 2B) between about 4 and about 16 inches. In other embodiments, the head channel has a width CW between about 7 and about 13 inches. In still other embodiments, the head channel has a width CW between about 8 and about 12 inches.

In some embodiments, at rest, the head channel extends into the head support to a depth (shown as CD in FIG. 2A) between about 0.2 inches and about 3.5 inches from a lateral-most surface of the face (26 or 28) in which the channel is defined. In other embodiments, the head channel extends to a depth CD between about 0.5 and about 2.5 inches into the head support. In still other embodiments, the head channel extends to a depth CD of between about 1 and about 1.5 inches into the head support.

Where the head support comprises the head channel, the channel may be disposed any suitable distance from the third and/or forth perimeter that allows the user to comfortably place the user's head and/or face in the channel when resting. In some embodiments, for instance, the FIG. 2B illustrates that the space (as shown by X in FIG. 2B) between the third perimeter 34 and the start 40 of the head channel 38 is between about 2 and about 8 inches. In other embodiments, the space X between the third perimeter and the start of the head channel is between about 3 and about 7 inches. In still other embodiments, the space X between the third perimeter and the start of the head channel is between about 4 and about 6 inches.

In another example of a suitable characteristic, when the head support is at rest, the support optionally comprises at least one ATMS (air tubing management system) notch. While an ATMS notch may serve any suitable purpose, in some instances, the ATMS notch allows the user to wear a facemask (e.g., an oxygen mask, a CPAP mask, etc.) and to rest the side of the user's face against the head support without having the support press against the facemask.

The ATMS notch can be any suitable shape, including, being rounded, square, polygonal, etc. In one embodiment, FIG. 2C shows that from a face view, the ATMS notch 42 can be defined in the head support 20 as a semi-circular void.

The ATMS notch can also have any suitable dimensions that allow it to fulfill its intended purposes. By way of example, the ATMS notch 42 can have any suitable width (as shown by FW in FIG. 2C). Indeed, in some embodiments, the ATMS notch has a width FW between about 2 and about 18 inches. In other embodiments, the ATMS notch has a width FW between about 5 and about 15 inches. In still other embodiments, the ATMS notch has a width FW between about 8 and about 12 inches. In another example, the ATMS notch can extend in from a perimeter of the head support to any suitable depth (shown as FD in FIG. 2C). In some embodiments, the ATMS notch has a depth FD of between about 1 and about 10 inches. In other embodiments, the ATMS notch extends to depth FD of between about 2.5 and about 8.5 inches. In still other embodiments, the ATMS notch extends to a depth FD of between about 3 and about 7 inches.

Additionally, the ATMS notch can be disposed in any suitable location. By way of example, FIG. 2C shows an embodiment in which the ATMS notch 42 is defined at the first perimeter 30. Similarly, FIG. 2D shows an embodiment in which the ATMS notch 42 is defined in both the first 30 and the second 32 perimeters of the head support 20. In such an embodiment, the user can rest on either the right or the left side without pressing a facemask against the head support.

In still another example of a suitable characteristic, when the head support is at rest, the support optionally comprises at least one shoulder notch. While the shoulder notch can serve any suitable purpose, in some cases, the shoulder notch allows the user to place the user's head on the head support (e.g., so the head channel is cradling the user's head) without requiring the user's shoulder to also be positioned on top of the head support. In this manner, the shoulder notch may help keep the user's neck straight when the user is lying in a side position.

The shoulder notch can have any suitable dimensions that allow it to function as intended. In one example, FIG. 2D shows the shoulder notch 44 can have any suitable length SL. In some embodiments, the shoulder notch has a length SL between about 5 and about 24 inches. In other embodiments, the shoulder notch has a length SL between about 10 and about 20 inches. In still other embodiments, the shoulder notch has a length SL between about 13 and about 17 inches. In another example, FIG. 2D shows the shoulder notch 44 can extend any suitable depth SD into the head support 20 from one of the support's perimeters (e.g., the third perimeter 34). In some embodiments, the shoulder notch extends to a depth SD of between about 1 and about 5 inches. In other embodiments, the shoulder notch extends to a depth SD of between about 1.5 and about 3.5 inches. In still other embodiments, the shoulder notch extends to a depth SD of between about 2 and about 3 inches.

The shoulder notch can be disposed in any suitable location that allows it to function as intended. In one example, FIG. 2D shows the shoulder notch 44 is defined at the third perimeter 34. In another example (not shown) the shoulder notch is defined at both the third and the fourth perimeters. In still another example, FIG. 2D shows the shoulder notch 44 can begin at any suitable distance SX from the first perimeter 30. Indeed, in some embodiments, the shoulder notch begins at the first perimeter. In other embodiments, the shoulder notch begins at a distance SX of between about 0.5 and about 8 inches from the first perimeter. In still other embodiments, the shoulder notch begins at a distance SX of between about 1 and about 6 inches from the first perimeter.

With respect to the torso support, this support can comprise any suitable characteristic that allows it to support a portion of the user's body that is between the user's neck and bottom (e.g., the user's back, chest, arms, stomach, etc.) when the user's head is positioned on the head support and the torso support is disposed near the user's torso. For instance, the torso support can comprise any suitable material, including but not limited to foam, natural fiber, synthetic fiber, down, air, fluid, other suitable pillow fillers, and combinations thereof. In some preferred embodiments, the torso support comprises the first type of foam (e.g., polyethylene foam) used in the main core of a foam head support.

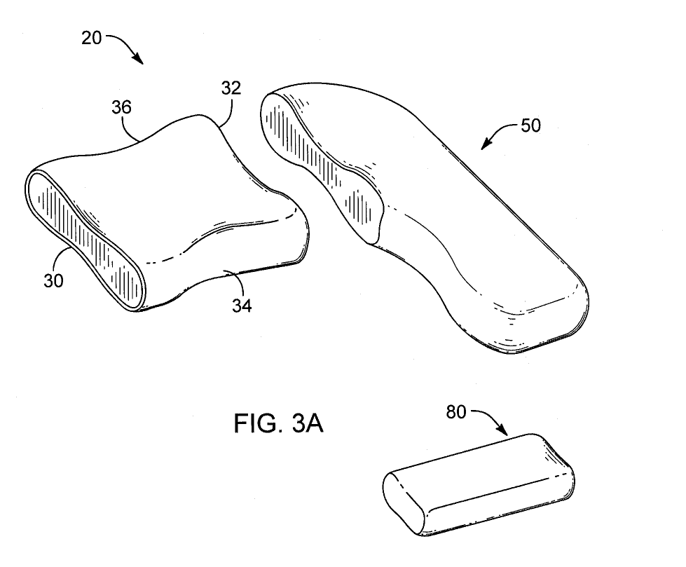

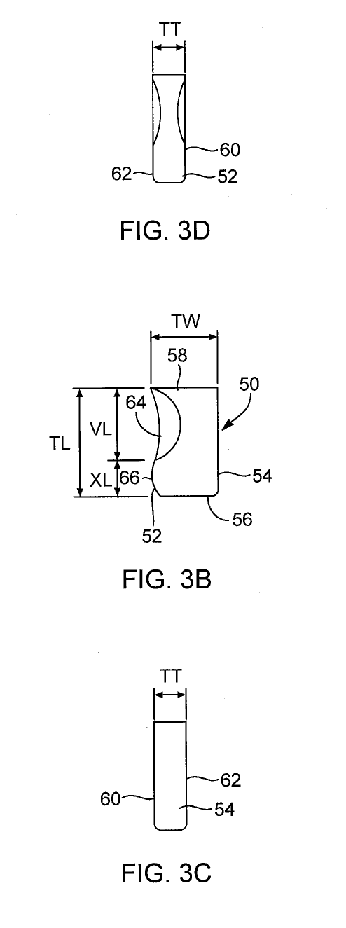

The torso support can be disposed with any suitable relation to the head support. For example, FIG. 1A shows an embodiment in which the torso support 50 abuts (or can be integrally formed as a single piece with the head support at) the head support's third perimeter 34. In this example, FIG. 1 shows that the head support 20 and torso support 50 have a substantially L-shaped appearance when connected. In other words, in FIG. 1A, the torso support 50 extends substantially perpendicular to the head support 20. In another example of a suitable relation between the head support 20 and the torso support 50. FIG. 3A shows another embodiment in which the torso support 50 abuts (or can be integrally formed as a single piece with the head support at) the second perimeter 32. In this example, the head support 20 and torso support 50 also connect to form a substantially L-shaped cushion.

The torso support can have any suitable dimensions that allow it to function as intended. For instance, FIG. 3B shows that the torso support 50 can have any suitable width TW between its first 52 and second 54 perimeters. In some embodiments, the torso support has a width TW of between about 2 and about 16 inches. In other embodiments, the torso support has a width TW of between about 4 and about 12 inches. In still other more preferred embodiments, the torso support has a width TW of between about 5.5 and about 10 inches.

FIG. 3B further shows the torso support 50 can have any suitable length TL between its third 56 and forth 58 perimeters (or between its third perimeter 56 and the third perimeter 34 of the head support 20). In some embodiments, the torso support has a length TL of between about 8 inches and about 36 inches. In other embodiments, the torso support has a length TL of between about 11 and about 19 inches. In still other embodiments, the torso support has a length TL of between about 13 and about 17 inches.

FIGS. 3C and 3D illustrate that the torso support 50 can also have any suitable thickness TT between its front face 60 and its back face 62. In some embodiments, the torso support has a thickness TT between about 1 and about 8 inches. In other embodiments, the torso support has a thickness TT between about 3 and about 7 inches. In still other embodiments, the torso support TT has a thickness of between about 4 and about 6.5 inches.

In some embodiments, when the torso support is at rest, a perimeter of the support defines concave portion and/or a convex portion. By way of illustration, FIG. 3B shows an embodiment in which the torso support 50 comprises both a concave portion 64 and a convex portion 66 that are defined at a first perimeter 52 of the support 50. Additionally, FIG. 3B shows that where the torso support 50 comprises a concave portion 64 and a convex portion 66, the concave portion 64 is preferably disposed closer to the head support (not shown in FIG. 3B) than is the convex portion 66. Thus, while the concave portion is configured to support the user's upper back (e.g., thoracic region), the convex portion is configured to support the user's lower back (e.g., lumbar region).

FIG. 3B shows that, at rest, the concave portion 64 can be any suitable length VL. In some embodiments, the concave portion has a length VL between about 4 and about 18 inches. In other embodiments, the concave portion has a length VL between about 6 and about 14 inches. In still other embodiments, the concave portion has a length VL between about 8 and about 12 inches.

FIG. 3B further shows that, at rest, the convex portion 66 can be any suitable length XL. In some embodiments, the convex portion has a length XL between about 2 and about 9 inches. In other embodiments, the convex portion has a length XL between about 3 and about 7 inches. In still other embodiments, the convex portion has a length XL between about 4 and about 6 inches.

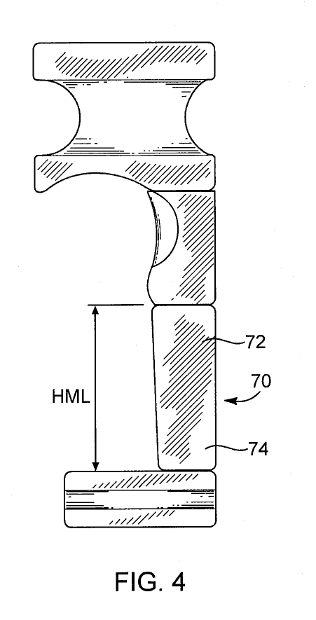

Referring now to the hinge member, the hinge member may comprise virtually any suitable and flexible connector that is more flexible than the torso support and that allows the leg support (discussed below) to be movably connected to the torso support. Accordingly, the hinge member may allow the user to easily adjust the placement of the leg support and to keep the leg support in the adjusted position without requiring the user to hold and maintain the leg support in the adjusted position. Some examples of suitable hinge members comprise a support casing, a stuffed compartment, rope, a strap, ribbon, and/or another connector that is more flexible than the torso member and that movably connects the torso support and the leg support. In some preferred embodiments, as shown in FIG. 4, a hinge member 70 comprises a compartment 72 that contains a filling 74 (e.g., natural fibers, synthetic fibers, down, etc.).

The hinge member can have any suitable characteristic that allows it to function as intended. For instance, FIG. 4 shows that the hinge member can have any suitable length HML that allows it to function as intended. In some embodiments, the hinge member has a length HML between about 12 and about 32 inches. In other embodiments, the hinge member has a length HML between about 17 and about 27 inches. In still other embodiments, the hinge member has a length HML between about 20 and about 24 inches.

With respect to the leg support, this support can comprise any suitable characteristic that allows it to cushion one or more portions of the user's legs (e.g., the user's thighs, knees, shins, ankles, feet, etc.). For instance, the leg support can comprise any suitable material, including, but not limited to, foam, natural fiber, synthetic fiber, down, air, fluid, other suitable pillow fillers, and combinations thereof. In some preferred embodiments, the leg support comprises the first type of foam (e.g., polyethylene foam) used in the main core of a foam head support.

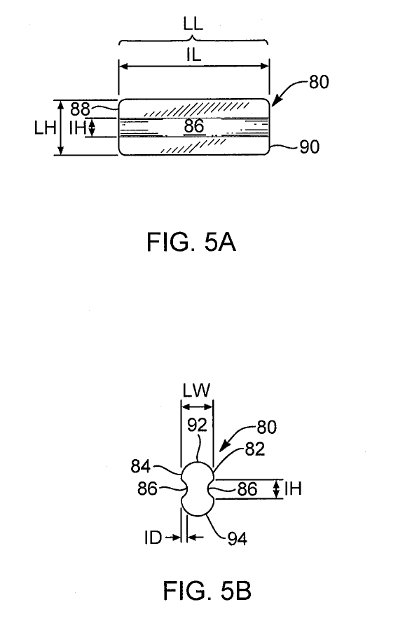

As another example of a suitable characteristic of the leg support, from its face view, the leg support may be any suitable shape, including, but not limited to, rectangular, square, circular, elliptical, polygonal, irregular, etc. FIG. 5A shows that, in a currently preferred embodiment, from its face view, the leg support 80 is substantially shaped as an elongated rectangle.

FIG. 5A shows the leg support 80 can have any suitable length LL that allows it to function as intended. In some embodiments, the leg support has a length LL between about 6 and about 36 inches. In other embodiments, the leg support has a length LL between about 15 and about 25 inches. In still other embodiments, the leg support has a length LL between about 17 and about 23 inches.

FIG. 5A shows the leg support 80 can have any suitable height LH that allows it to perform its intended purposes. In some embodiments, the leg support has a height LH between about 2 and about 14 inches. In other embodiments, the leg support has a height LH between about 4 and about 10 inches. In still other embodiments, the leg support has a height LH between about 6 and about 9 inches.

FIG. 5B shows the leg support 80 may have any suitable width LW between the lateral-most edge of its front face 82 and the opposite lateral-most edge of its back face 84. In some embodiments, the leg support has a width LW between about 1 and about 10 inches. In other embodiments, the leg support has a width LW between about 2 and about 7 inches. In still other embodiments, the leg support has a width LW between about 3.5 and about 5.5 inches.

In some embodiments, the leg support comprises at least one indentation that is configured to receive one or more portions of the user's legs. While this indentation may perform any suitable function, in some embodiments, the indentation helps to receive and hold a portion of the user's leg in a manner that makes it easy for the leg support to be maintained between the user's legs. FIG. 5B shows an embodiment in which the leg support 80 comprises an indentation 86 its front face 82 and its back face 84.

Where the leg support comprises an indentation, the indentation can comprise any suitable characteristic. For instance, the indentation can be any suitable size. By way of illustration, FIG. 5A shows the indentation 86 can be any suitable length IL. In some embodiments, the indentation has a length IL between about 1.5 and about 6 inches. In other embodiments, the indentation has a length IL between about 5 and about 18 inches. In still other embodiments, FIG. 5A shows the indentation 86 has a length IL that substantially extends between the leg support's first 88 and second 90 perimeter.

FIGS. 5A and 5B show the indentation 86 can have any suitable height IH. Indeed, in some embodiments, the indentation has a height between about 0.5 and about 7 inches. In other embodiments, the indention has a height IH between about 1 and about 3.5 inches. In still other embodiments, the indention has a height IH between about 1.5 and about 3 inches.

FIG. 5B also shows that the indentation 86 may extend any suitable depth ID in to the support 80 from the lateral-most surface of face (e.g., 82 or 84) in which it is defined. In some embodiments, the indentation has a depth ID of between about 0.5 and about 3 inches. In other embodiments, the indentation has a depth ID of between about 1 and about 2.5 inches. In still other embodiments, the indentation has a depth ID of between about 1 and about 2 inches.

FIG. 5B also shows that the top edge 92 and the bottom edge 94 of the leg support may be rounded. Thus, FIG. 5B shows that where the leg support 80 comprises the indentation 86 on the leg supports front face 82 and back face 84, the leg support 80 can have a peanut-shaped appearance from a side view.

In addition to the aforementioned components, the cushioning device may comprise any other suitable component, including, but not limited to, a support casing and a pillow case. Where the cushioning device comprises a support casing, the casing may serve any suitable purpose, including connecting the various supports together.

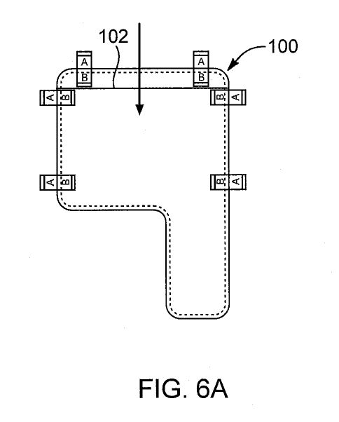

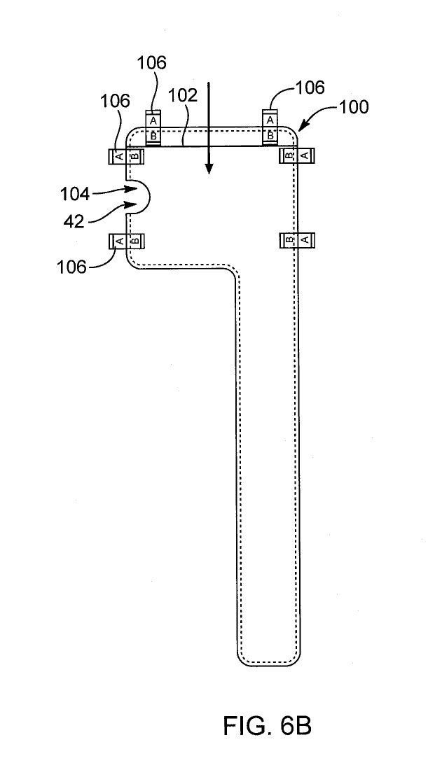

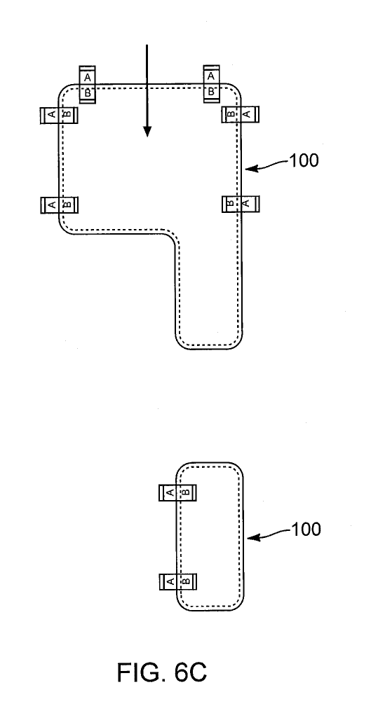

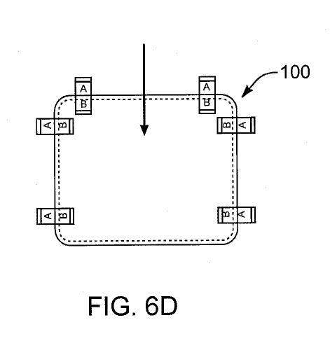

The casing can have any suitable characteristic that allows it to cover one more supports. Indeed, in some embodiments, the casing has a separate compartment for each support. In other embodiments, the casing has at least one compartment that holds more than one support. In still other embodiments, the casing comprises one or more openings, including, but not limited to, a flap, a zippered opening, an opening with hook-and-loop fasteners, or any other opening through which a support of the cushion device can be removed. Where the casing comprises an opening, the user can remove and replace/interchange one or more supports. For instance, the user can use the opening to replace the head support with a conventional bed pillow.

Where the cushioning device comprises a pillow case, the pillow case can comprise any characteristic that allows it to cover one or more supports and/or the casing of the cushioning device. In one example, FIG. 6A shows the pillow case comprises one or more openings (e.g., a flap opening 102). In another example, the pillow case is configured to hold a single support. In still another example, the pillow case is configured to cover multiple supports. For instance, FIG. 6A shows an embodiment in with the pillow case 100 is configured to hold the head and torso supports (not shown in FIG. 6A). In still another example, the pillow case is configured to cover all of the elements of the cushioning device. An embodiment of such a pillow case 100 is illustrated in FIG. 6B. In yet another embodiment, FIG. 6C shows the pillow case 100 is configured to cover the leg support (not shown in FIG. 6C) as a discrete support. In still another embodiment, FIG. 6D shows the pillow case 100 is configured to cover the head support (not shown in FIG. 6D) as a discrete support.

In yet another example of a suitable characteristic, the pillow case can comprise darts or otherwise be shaped to fit the contours (e.g., the ATMS notch, shoulder notch, convex portion, concave portion, etc.) of the supports. By way of example, FIG. 6B shows an embodiment in which the pillow case 100 comprises a dart 104 for the ATMS notch 42.

In another example, the pillow case comprises ATMS connectors for holding tubing (e.g., facemask tubing) in place. In such embodiments, the pillow case can help keep tubing in a convenient place and thereby reduce the likelihood of the user being choked or annoyed by the tubing. Some non-limiting examples of suitable ATMS connector comprise straps with hook-and-loop fasteners, buckles, snaps, etc.; ties; and other objects that can selectively retain and release tubing from the pillow case. By way of example, FIG. 6B shows an embodiment in which the pillow case 100 comprises a plurality of straps 106 comprising hook-and-loop fasteners. In this example, the straps 106 can be used to secure tubing (e.g., for a facemask) toward the top of the cushion device.

The described cushioning device can be modified in any suitable manner that allows it to function as intended. In some embodiments, the size of the various components are of the cushion device are tailored to users of particular sizes. For instance, the cushion device can be tailored for infants, toddlers, children, short users, tall users, and practically any other sized user.

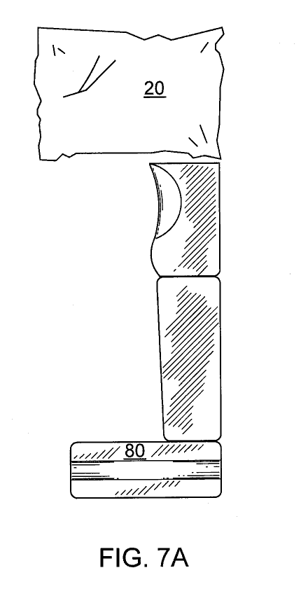

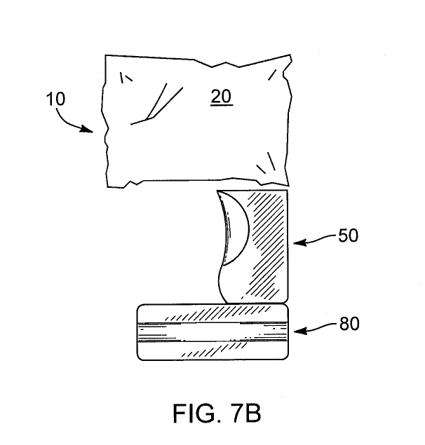

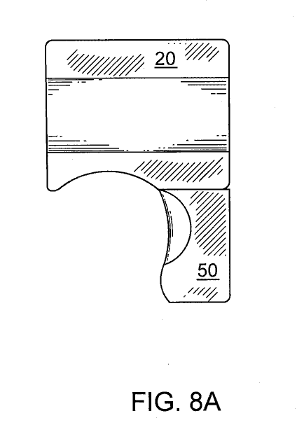

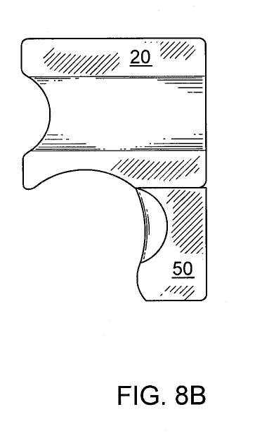

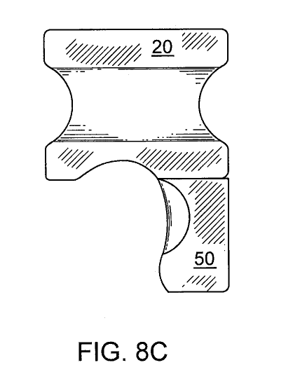

As another example of a suitable modification, FIGS. 7A and 7B show some embodiments in which the head support 20 comprises a conventional pillow (see also FIG. 1G). As another example, FIG. 7B shows the cushioning device 10 need not comprise a hinge member (not shown in FIG. 7B). Accordingly, the leg support 80 can be used separate from the head support 20 or need not be used at all. In still another example, FIGS. 8A through 8C show the head support 20 and torso support 50 can be used together (as discrete or as integrally-connected supports) without the leg support and hinge member (neither of which are shown in FIGS. 8A-8C).

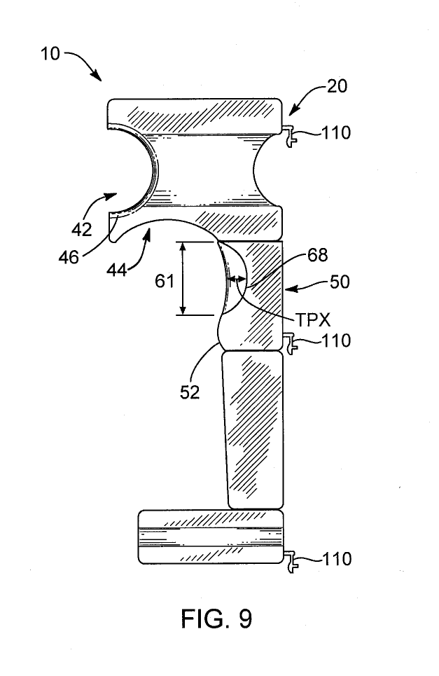

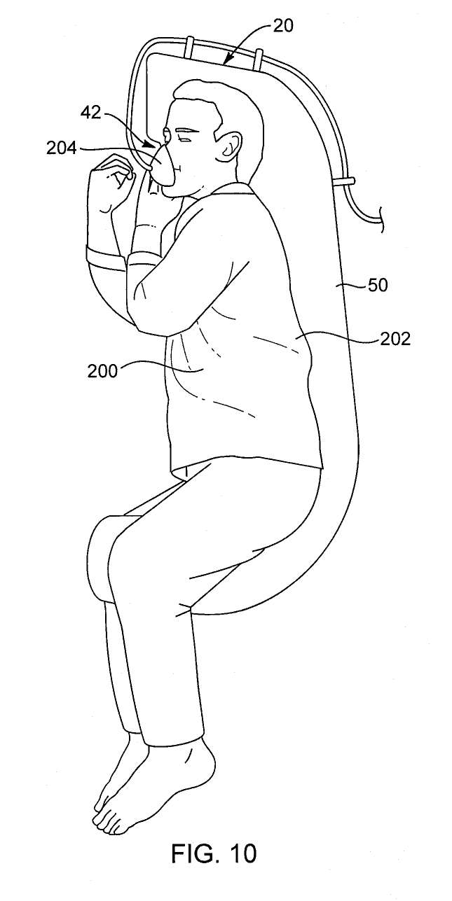

In some embodiments, one or more perimeters on one or more faces, of one or more supports, are tapered or rounded to increase the user's comfort level when the user contacts those perimeters. Any perimeter of the supports can be tapered or rounded. In one example, FIG. 9 shows the ATMS notch 42 having a tapered perimeter 46. In another example, FIG. 9 shows the shoulder notch 44 having a tapered perimeter 48. In still another non-limiting example, FIG. 9 shows an embodiment in which the concave portion 64 of the torso support's first perimeter 52 comprises a tapered edge 68.

As with all other tapered edges, FIG. 9 shows the tapered edge 68 on the concave portion 64 can extend in its support (torso support 50) any suitable distance TPX. In some embodiments, the tapered perimeter on the concave portion extends into the torso support to a distance TPX between about 0.5 and about 6 inches. In other embodiments, the tapered perimeter on the concave portion extends in to the torso support by a distance TPX of between about 1 and about 4.5 inches. In still other embodiments, the tapered perimeter on the concave portion extends into the torso support to a distance between about 1.5 and about 3.5 inches.

In other embodiments, one or more of the supports (e.g., the head, torso, and/or leg support) are configured to be selectively inflated and deflated. In such embodiments, the supports, individually, in combination, or as a single integrated device, can be easily deflated, transported, and then inflated again. Similarly, in such embodiments, the supports can be inflated to different pressures so that each support has a desired firmness.

Where one or more of the support sections is selectively inflatable, the supports can comprise any suitable characteristic that allows them to function as intended. In one example, FIG. 9 shows that one or more supports can comprise a closable valve 110. In another example, each inflatable support can comprise any suitable characteristic that provides the support with the desired features (e.g., head channels, ATMS notches, shoulder notches, indentations, shape, thickness, etc.). For instance, the inflatable compartments can include seams, internal restraints, and other components that help the supports retain their desired shapes when inflated.

In other embodiments, one or more layers of memory foam are disposed on other supports besides just the described head support comprising a main core. In one example, an inflatable head support comprises a layer of memory foam on the head support's front or back face. In another example, one or more portions of the back support and/or the leg support—regardless of the materials from which those supports are made—comprise a layer of memory foam.

In alternative embodiments, the cushioning device includes means to provide heat to the different supports. Some non-limiting examples of such heating means include pockets to hold hot water bottles, heating coils, and so forth. While such heating means can perform a variety of functions, in some cases, the heating means help to alleviate pain (e.g., muscle pain, arthritis pain, etc.), to calm restless leg syndrome, to promote increased circulation, and to further encourage relaxation and restful sleep.

In still other embodiments, the cushioning device includes means to provide sound (or music), massage, vibration, and or scents or aromas to the different support sections.

In still other embodiments, the cushioning device comprises means to sense certain physical functions of the user, such as breathing effort, respiratory effort, respiratory events, apnea or hyponea events, neural activity, snoring events, snoring sound intensity, vibrations or movement in limbs, temperature, blood pressure, pulse rate, and oxygen and CO₂ saturation levels in the user. These means can include heart rate monitors, blood pressure monitors, electroencephalography sensors, neural sensors, physical sensors, eye movement sensors, nasal or mouth breathing sensors, blood composition sensors, muscle sensors, or other polysomnographic sensors. In some instances, the cushioning device also has means to record the sensors or display results from the sensors to others or to the user.

In yet other embodiments, the cushioning device has means to monitor the user and allow for self-assessment, third-party, or professional assessment. In such embodiments, the cushioning device can relay results in any suitable manner, including, but not limited to, through means of a physical, telecommunication, or internet system, which may relay the sensed results of the user whereby they are forwarded on to a medical site, or computerized medical center to allow for consistent, intermittent, or alarm-based medical monitoring. For example, in the event that the user's sleep apnea creates dangerously low oxygen levels or the user's detected recordings indicates the user is suffering from a serious medical condition, such as heart attack or stroke, the sensing and diagnostic system may contact emergency or non-emergency medical services.

The described cushioning device can be made in any suitable manner. For example, the various support sections can be sewn, cut, shaved, molded, extruded, stamped, stuffed, heat-welded (in the case of inflatable supports), or otherwise formed to have the desired characteristics. Similarly, the casing and/or pillow case can be sewn, cut, stamped, heat-welded, or otherwise formed to cover the supporting sections.

The described cushioning device can also be used in any suitable manner. For example, FIG. 10 shows one embodiment in which the user 200 is laying on the cushioning device 10 with the user's back 202 towards the torso support 50. In this example, the user's facemask 204 is disposed adjacent to the ATMS notch 42 to allow the user 200 to lie on the user's right side without pressing the facemask 204 against the head support 20. In another embodiment, which is not shown, the user lays with the user's face towards torso support. In still another embodiment, which is not illustrated, the user could flip the cushioning device over and rest in a left-side position with the user's back towards the torso support.

Thus, as discussed herein, the embodiments of the present invention embrace cushioning devices that encourage a user to sleep in a side position as opposed to a supine or prone position. Embodiments of the present invention may be embodied in other specific forms without departing from its spirit or essential characteristics. The described embodiments and examples are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A head support pillow, comprising:
a front face;
a back face disposed substantially opposite to the front face;
a first perimeter;
a second perimeter disposed substantially opposite to the first perimeter;
a third perimeter; and
a forth perimeter disposed substantially opposite to the third perimeter, wherein when the pillow is at rest, the first perimeter defines a first air tubing management system (“ATMS”) notch that extends into the pillow, towards the second perimeter.

2. The head support pillow of claim 1, wherein the second perimeter defines a second ATMS notch that extends into the pillow, towards the first perimeter.

3. The head support pillow of claim 1, wherein the third perimeter defines a shoulder notch that extends into the pillow, towards the forth perimeter.

4. The head support pillow of claim 1, wherein the pillow comprises a main core comprising a first foam material with a first level of firmness, and the front face comprises a second foam material having a second level of firmness that is softer than the first level of firmness.

5. The head support pillow of claim 1, wherein the pillow comprises a head channel that indents into the front face.

6. The head support pillow of claim 1, wherein a torso support extends from the head support pillow to form a cushioning device with an L-shaped appearance.

7. A cushioning device, comprising:
a head support pillow; and
a torso support extending from the head support so the cushioning device has a substantially L-shaped appearance, the torso support comprising:
a first perimeter disposed substantially opposite to a second perimeter, the first perimeter comprising a concave portion and convex portion, wherein the concave portion is disposed closer to the head support than the convex portion.

8. The cushioning device of claim 7, wherein head support comprises a first edge disposed substantially opposite to a second edge, and wherein the first edge, at rest, defines a first ATMS notch that extends into the head support pillow, towards the second edge.

9. The cushioning device of claim 7, wherein the head support comprises:
a front face defining a first head channel that extends into the head support, towards a back face of the head support;
the back face defining a second head channel that extends into the head support, towards the front face;
a main core comprising a first type of foam; and
a layer of memory foam disposed on the front face.

10. The cushioning device of claim 8, wherein the second defines a second ATMS notch that extends into the head support pillow, towards the first edge.

11. The cushioning device of claim 7, wherein the head support comprises a first edge disposed substantially opposite to a second edge, a third edge disposed substantially perpendicular to the first edge and substantially opposite to a forth edge, wherein a length of the torso support runs substantially perpendicular to a length of the third edge, and wherein the third edge comprises a shoulder notch that extends into the head support, towards the forth edge.

12. The cushioning device of claim 7, wherein the head support and the cushioning support are selectively inflatable and deflatable.

13. A therapeutic cushioning device, comprising:
a head support;
a torso support extending from the head support to form an L-shaped cushion; and
a leg support, wherein the torso support comprises a first perimeter disposed substantially opposite to a second perimeter, the first perimeter defining a concave portion and a convex portion, the concave portion being disposed closer to the head support than the convex portion, and the leg support comprises a front face and a back face, the front face and the back face each defining an indentation.

14. The cushioning device of claim 13, further comprising a hinge member that movably connects the leg support to the torso support, wherein the hinge member is more flexible than the torso support.

15. The cushioning device of claim 13, wherein, at rest, the head support comprises a first edge disposed substantially opposite to a second edge, and a third edge disposed substantially perpendicular to the first edge and substantially opposite to a fourth edge, and wherein the first edge defines an ATMS notch that extends into the head support, towards the second edge.

16. The cushioning device of claim 15, wherein, at rest, the head support comprises a first edge disposed substantially opposite to a second edge, and a third edge disposed substantially perpendicular to the first edge and substantially opposite to a fourth edge, and wherein the third edge defines a shoulder notch that extends into the head support, towards the forth edge.

17. The cushioning device of claim 13, wherein, at rest:
the head support comprises a first edge disposed substantially opposite to a second edge, and a third edge disposed substantially perpendicular to the first edge and substantially opposite to a fourth edge;
the first edge defines a first ATMS notch that extends into the head support towards the second edge;
the second edge defines a second ATMS notch that extends into the head support towards the first edge; and
the third edge defines a shoulder notch that extends into the head support towards the fourth edge.

18. The cushioning device of claim 13, wherein the head support, the torso support, and the leg support are selectively inflatable and deflatable.

19. The cushioning device of claim 18, wherein a cushion selected from the head support, the torso support, the leg support, and combinations thereof comprises a layer of memory foam.

20. The cushioning device of claim 13, further comprising a pillow case having means for securing tubing to the pillow case.
